(19) United States
(12) Patent Application Publication
(10) Pub. No.: US 2025/0031984 A1
(43) Pub. Date: Jan. 30, 2025

(54) BLOOD FLOW RESTRICTIVE CUFF WITH INTEGRATED BLOOD FLOW SENSOR

(71) Applicant: KCI Manufacturing Unlimited Company, Westmeath (IE)

(72) Inventors: Jonathan G. REHBEIN, Saint Paul, MN (US); Javier GONZALEZ, Saint Paul, MN (US); Christopher J. CARROLL, Saint Paul, MN (US)

(21) Appl. No.: 18/717,322

(22) PCT Filed: Nov. 17, 2022
(86) PCT No.: PCT/IB2022/061069

§ 371 Date: Jun. 6, 2024

Related U.S. Application Data
(60) Provisional application No. 63/291,260, filed on Dec. 17, 2021.

(51) Int. Cl. A61B 5/0225; A61B 5/00; A61B 5/024; A61B 5/026

(52) U.S. Cl. CPC A61B 5/02255; A61B 5/02416; A61B 5/0261; A61B 5/6843

(57) ABSTRACT

A cuff for measuring blood flow through arteries of a limb may include an interior surface, a bladder, and a blood flow sensor. The interior surface of the cuff is configured to be secured around the limb. The bladder is configured to be fluidly coupled to a source of positive pressure. The bladder is further be adapted to inflate in response to application of a positive pressure and restrict blood flow through the arteries of the limb when inflated. The blood flow sensor is coupled to the interior surface and disposed between the interior surface and the limb. The blood flow sensor is configured to measure blood flow through the arteries of the limb.

### CROSS-REFERENCE TO RELATED APPLICATIONS

This application is a U.S. National Stage Entry of PCT/IB2022/061069, filed Nov. 17, 2022, which claims the benefit of priority to U.S. Provisional Application No. 63/291,260, filed on Dec. 17, 2021, each of which is incorporated herein by reference in its entirety.

### TECHNICAL FIELD

The invention set forth in the appended claims relates generally to blood flow restrictive therapy and more particularly, but without limitation, to systems, methods, and apparatuses for monitoring blood flow through a limb during blood flow restrictive therapy.

### BACKGROUND

Clinical studies and practice have shown that when training in a hypoxic environment, increased muscle strength and growth can be achieved when compared to training in a non-hypoxic environment. A hypoxic environment can be achieved by completing high intensity exercise. However, some people cannot complete high intensity workouts or exercises because the high intensity nature of the workouts may be too much for their bodies to handle. More specifically, people who are recovering from an injury or a surgery may need to complete lower intensity workouts to rebuild their strength. Blood flow restrictive therapy may be used to create a hypoxic environment without the need for high intensity activity. Blood flow restrictive therapy has been shown to mimic the effects of high intensity activity and aid in rehabilitation as well as general strength training efforts.

While the clinical benefits of blood flow restrictive therapy are widely known, improvements to systems, components, and processes may benefit users, healthcare providers, and patients.

### BRIEF SUMMARY

New and useful systems, apparatuses, and methods for monitoring blood flow through a limb during blood flow restrictive therapy are set forth in the appended claims. Illustrative embodiments are also provided to enable a person skilled in the art to make and use the claimed subject matter.

For example, in some embodiments, a cuff for measuring blood flow through arteries of a limb is described. The cuff can include an interior surface, a bladder, and a blood flow sensor. The interior surface of the cuff can be configured to be secured around the limb. The bladder can be configured to be fluidly coupled to a source of positive pressure and restrict blood flow through the arteries of the limb when inflated. The blood flow sensor can be coupled to the interior surface of the cuff and can be disposed between the interior surface and the limb. The blood flow sensor can be configured to measure blood flow through the arteries of the limb.

In some example embodiments, the blood flow sensor can be a photoplethysmography (PPG) sensor with an LED and a photodiode. The PPG sensor can be in an absorptive mode with the LED and the photodiode disposed on opposite side of the interior surface of the cuff. In other embodiments, the PPG sensor can be in a reflectance mode with the LED and the photodiode aligned on one side of the interior surface of the cuff. The PPG sensor can be further configured to measure oxygen levels of blood in the limb.

In other example embodiments, the blood flow sensor can be an optical sensor with an LED and a photodiode. The optical sensor can be in a reflectance mode with the LED and the photodiode aligned on one side of the interior surface of the cuff.

In some embodiments, the source of positive pressure can be adjusted manually. In other embodiments, the blood flow sensor can be communicatively coupled to a controller. The controller can be configured to receive data from the blood flow sensor and to adjust the source of positive pressure in response to the data received from the blood flow sensor.

A system for measuring blood flow through arteries of a limb is also described herein. The system can include a cuff and a source of positive pressure. The cuff can include an interior surface, a bladder, and a blood flow sensor. The interior surface of the cuff can be configured to be secured around the limb. The bladder can be configured to be fluidly coupled to a source of positive pressure and restrict blood flow through the arteries of the limb when inflated. The blood flow sensor can be coupled to the interior surface of the cuff and can be disposed between the interior surface and the limb. The blood flow sensor can be configured to measure blood flow through the arteries of the limb. The source of positive pressure can be configured to be fluidly coupled to the bladder of the cuff and to inflate the bladder of the cuff when actuated.

In some example embodiments, the system can further include a controller that can be communicatively coupled to the blood flow sensor and the source of positive pressure. The controller can be configured to receive signals from the blood flow sensor and to actuate the source of positive pressure in response to the signals from the blood flow sensor. In some embodiments, the controller may further be configured to receive therapy input values. The controller may actuate the source of positive pressure in response to the therapy input values.

In some example embodiments, the system can further include a therapy unit that can be configured to output values indicative of the blood flow through the arteries of the limb. In still other example embodiments, the blood flow sensor can be further configured to measure an oxygen level of blood in the arteries of the limb.

A method of restricting blood flow through arteries of a limb is also described herein. The method can include providing a cuff wherein the cuff can include an interior surface, a bladder, and a blood flow sensor. The interior surface of the cuff can be configured to be secured around the limb. The bladder can be configured to be fluidly coupled to a source of positive pressure and restrict blood flow through the arteries of the limb when inflated. The blood flow sensor can be coupled to the interior surface of the cuff and can be disposed between the interior surface and the limb. The blood flow sensor can be configured to measure blood flow through the arteries of the limb. The method can further include securing the cuff around the limb, sensing, with the blood flow sensor, a baseline arterial blood flow through the arteries of the limb, coupling the bladder to the source of positive pressure, and inflating the bladder with the source of positive pressure until the blood flow sensor senses a restricted arterial blood flow. The restricted arterial blood flow can indicate that the blood flow through the arteries of the limb is completely occluded. The method can further include adjusting the bladder to reach a target arterial blood flow. The target arterial blood flow can be between the baseline arterial blood flow and the restricted arterial blood flow.

In some example embodiments, adjusting the bladder to reach the target arterial blood flow can include adjusting the bladder until the target arterial blood flow is reached. In other embodiments, adjusting the bladder to reach the target arterial blood flow can include receiving, with the controller, a signal from the blood flow sensor that the restricted arterial blood flow has been reached. Adjusting the bladder to reach the target arterial blood flow can further include sending, with the controller, a signal to the source of positive pressure to stop the source of positive pressure from delivering positive pressure to the bladder of the cuff. Adjusting the bladder to reach the target arterial blood flow can further include releasing fluid from the bladder of the cuff until the target arterial blood flow is reached. In some embodiments, releasing fluid from the bladder of the cuff until the target arterial blood flow is reached can include sending, with the controller, a signal to the source of positive pressure to switch the source of positive pressure to a vacuum mode and removing fluid from the bladder of the cuff with the source of positive pressure until the target arterial blood flow is reached.

In some example embodiments, the method can further include sensing, with the blood flow sensor, an oxygen level of blood in the arteries of the limb. The method can further include modifying the target arterial blood flow to incorporate the oxygen level of blood in the arteries of the limb.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a functional block diagram of a cuff system for providing blood flow restrictive therapy to a limb;

FIG. 2 is a perspective view of the cuff system of FIG. 1 coupled to a limb of a user;

FIG. 3 is a partial sectional view of the cuff system of FIG. 1 with a bladder of a cuff of the cuff system in a resting state;

FIG. 4 is a partial sectional view of the cuff system of FIG. 1 with the bladder in an inflated state;

FIG. 5 is a partial sectional view of the cuff system of FIG. 1 in an operational state in an absorptive mode; and

FIG. 6 is a partial sectional view of the cuff system of FIG. 1 in an operational state in a reflectance mode; and

FIG. 7 is a block diagram of a method of restricting blood flow through an artery of a limb, according to an exemplary embodiment.

### DESCRIPTION OF EXAMPLE EMBODIMENTS

The following description of example embodiments provides information that enables a person skilled in the art to make and use the subject matter set forth in the appended claims, but it may omit certain details already well-known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

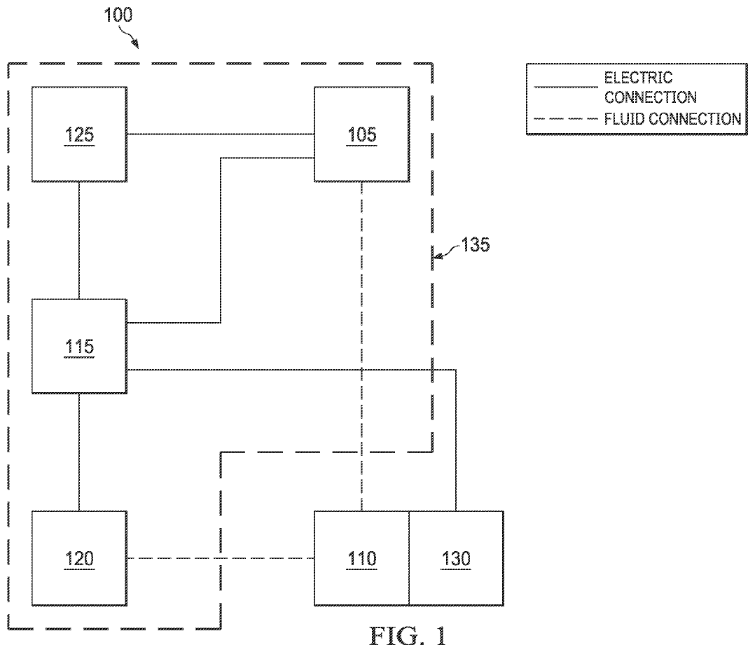

FIG. 1 is a simplified functional block diagram of an example embodiment of a cuff system 100 that can provide blood flow restrictive therapy to a limb in accordance with this specification.

The term “limb” in this context broadly refers to an extremity such as an arm or a leg. The cuff system 100 may be configured to measure and control blood flow through a limb during the course of a blood flow restrictive therapy.

The cuff system 100 may include a supply of positive pressure, such as a source of positive pressure 105, and one or more distribution components. A distribution component is preferably detachable and may be disposable, reusable, or recyclable. A cuff, such as a cuff 110 is an example of a distribution component that may be associated with some examples of the cuff system 100. A fluid conductor is another illustrative example of a distribution component. A “fluid conductor,” in this context, broadly includes a tube, pipe, hose, conduit, or other structure with one or more lumina or open pathways adapted to convey a fluid between two ends. Typically, a tube is an elongated, cylindrical structure with some flexibility, but the geometry and rigidity may vary. Moreover, some fluid conductors may be molded into or otherwise integrally combined with other components. Distribution components may also include or comprise interfaces or fluid ports to facilitate coupling and de-coupling other components.

The cuff system 100 may also include a regulator or controller, such as a controller 115. Additionally, the cuff system 100 may include sensors to measure operating parameters and provide feedback signals to the controller 115 indicative of the operating parameters. For example, the cuff system 100 may include a first sensor 120, a second sensor 125, and a blood flow sensor 130 coupled to the controller 115.

Some components of the cuff system 100 may be housed within or used in conjunction with other components, such as sensors, processing units, alarm indicators, memory, databases, software, display devices, or user interfaces that further facilitate therapy. For example, in some embodiments, the source of positive pressure 105 may be combined with the controller 115 and other components into a therapy unit 135.

In general, components of the cuff system 100 may be coupled directly or indirectly. For example, the source of positive pressure 105 may be directly coupled to the cuff 110. Coupling may include fluid, mechanical, thermal, electrical, communicative (such as wirelessly), or chemical coupling (such as a chemical bond), or some combination of coupling in some contexts. For example, the source of positive pressure 105 may be electrically or communicatively coupled to the controller 115 and may be fluidly coupled to one or more distribution components to provide a fluid path to the cuff 110. In some embodiments, components may also be coupled by virtue of physical proximity, being integral to a single structure, or being formed from the same piece of material.

A positive pressure supply, such as the source of positive pressure 105, may be a reservoir of air or may be a manual or electrically powered device. “Positive pressure” generally refers to a pressure greater than a local ambient pressure, such as the ambient pressure in a local environment external to a sealed therapeutic environment. In many cases, the local ambient pressure may also be the atmospheric pressure at which the limb is located. Unless otherwise indicated, values of pressure stated herein are gauge pressures. Common therapeutic ranges are between 50 mm Hg (6.67 kPa) and 350 mm Hg (46.67 kPa).

A controller, such as the controller 115, may be a microprocessor or computer programmed to operate one or more components of the cuff system 100, such as the source of positive pressure 105. In some embodiments, for example, the controller 115 may be a microcontroller, which generally comprises an integrated circuit containing a processor core and a memory programmed to directly or indirectly control one or more operating parameters of the cuff system 100. Operating parameters may include the power applied to the source of positive pressure, the pressure generated by the source of positive pressure 105, or the pressure distributed to the cuff 110, for example. The controller 115 is also preferably configured to receive one or more input signals, such as a feedback signal, and programmed to modify one or more operating parameters in response to the input signals.

In some embodiments, the controller 115 may receive and process data from one or more sensors, such as the first sensor 120, the second sensor 125, and the blood flow sensor 130. The controller 115 may also control the operation of one or more components of the cuff system 100 to manage the pressure delivered to the cuff 110. In some embodiments, controller 115 may include an input for receiving a desired target pressure or a desired target blood flow through the limb. The controller 115 may be programmed for processing data relating to the setting and inputting of the target pressure to be applied to the cuff 110. In some example embodiments, the target pressure may be a fixed pressure value set by an operator as the target negative pressure desired for therapy at a tissue site and then provided as input to the controller 115. The target pressure may vary depending on the type of limb, the type of injury or treatment, and the preference of the attending physician. After selecting a desired target pressure or target blood flow, the controller 115 can operate the source of positive pressure in one or more control modes based on the target pressure or the target blood flow and may receive feedback from one or more sensors to maintain the target pressure at the cuff 110 or the target blood flow through the limb.

Sensors, such as the first sensor 120, the second sensor 125, and the blood flow sensor 130, may be apparatuses operable to detect or measure a physical phenomenon or property, and generally provide a signal indicative of the phenomenon or property that is detected or measured. For example, the first sensor 120 and the second sensor 125 may be configured to measure one or more operating parameters of the cuff system 100. In some embodiments, the first sensor 120 may be a transducer configured to measure pressure in the cuff 110 and convert the measurement to a signal indicative of the pressure measured. The second sensor 125 may optionally measure operating parameters of the source of positive pressure 105, such as a voltage or current, in some embodiments. The blood flow sensor 130 may be configured to measure blood flow through a limb when the cuff 110 is secured around the limb. The signals from the first sensor 120, the second sensor 125, and the blood flow sensor 130, are operable as an input signal to the controller 115, but some signal conditioning may be appropriate in some embodiments. For example, the signal may need to be filtered or amplified before it can be processed by the controller 115. Typically, the signal is an electrical signal, but may be represented in other forms, such as an optical signal.

The cuff 110 can be generally adapted to partially or fully contact a limb. The cuff 110 may take many forms, and may have many sizes, shapes, or thicknesses, depending on a variety of factors, such as the type of treatment being implemented or the nature and size of a limb. In some embodiments, the cuff 110 may comprise or consist essentially of a material configured to be positioned around a limb. The cuff 110 may be comprised of a material that can stretch and be conformable while in contact with skin. In some embodiments, the cuff 110 may be comprised of an elastic material or a nylon material.

The cuff 110 may be adapted to receive positive pressure from a source such as the source of positive pressure 105 and distribute positive pressure through a bladder of the cuff 110. In some embodiments, a fluid path may be reversed or a secondary fluid path may be provided to facilitate removing fluid from the bladder of the cuff 110.

The width of the cuff 110 may vary according to the needs of the prescribed therapy. The width of the cuff 110 may need to be increased to accommodate a larger limb. In some embodiments, the width of the cuff 110 may be about 15 cm to achieve a desired blood flow restriction through the limb. The length of the cuff 110 may also vary according to needs of a prescribed therapy. For example, the length of the cuff 110 may be decreased to accommodate a smaller limb. Similarly, the length of the cuff 110 may be increased to accommodate a larger limb.

In operation, the cuff 110 may be placed around a limb. The source of positive pressure 105 may be coupled to the cuff 110 and may deliver positive pressure to the cuff 110 for the duration of the blood flow restrictive therapy. Positive pressure applied to the cuff 110 can apply pressure to the limb to completely restrict blood flow in the veins of the limb. The positive pressure may also partially restrict the blood flow through the arteries of the limb to facilitate pooling of blood in the limb. Pooling of blood in the limb may induce muscular fatigue and may create a hypoxic environment. A hypoxic environment may lead to the release of growth hormones which may lead to increased muscle strength and muscle mass.

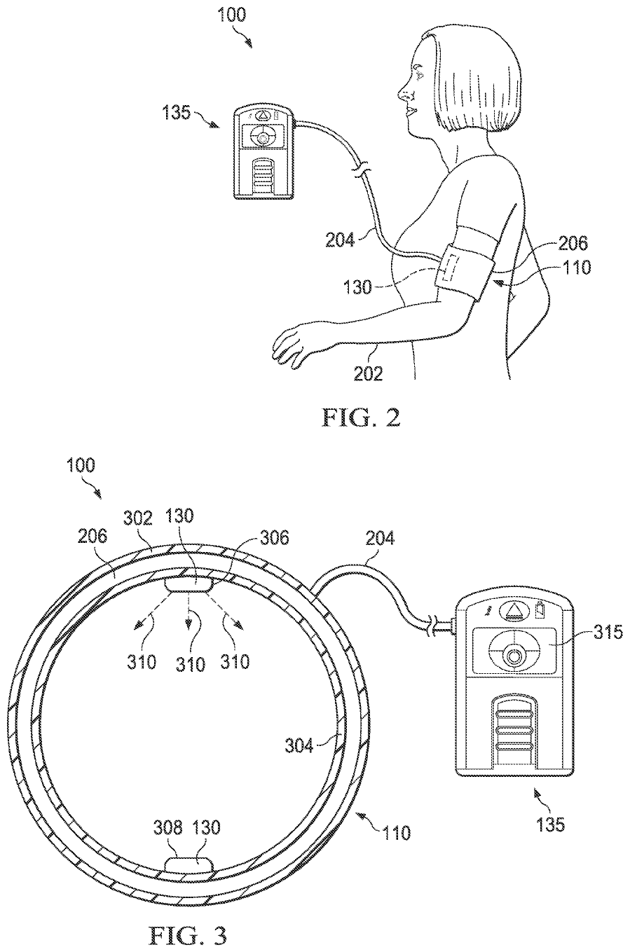

FIGS. 2 depicts an example embodiment of the cuff system 100 of FIG. 1 secured around a limb 202. The cuff system 100 may include the cuff 110 and the therapy unit 135 which may include the source of positive pressure 105 and the controller 115. In some embodiments, the therapy unit 135 may be a computer or another electronic device capable of outputting information and data associated with the cuff system 100. In other embodiments, the therapy unit 135 may include the source of positive pressure 105 and the controller 115 may be separated from the therapy unit 135 such that the controller 115 is wirelessly connected to the therapy unit 135. The cuff 110 may be secured around the limb 202 and may be coupled to the therapy unit 135 with a conduit 204. The cuff 110 may include an inflatable component or a bladder 206 that is inflatable and the blood flow sensor 130.

Referring to FIG. 3, the cuff 110 may have an exterior surface 302 and an interior surface 304 opposite the exterior surface 302. The interior surface 304 of the cuff 110 may be configured to be secured around the limb 202. In some embodiments, the cuff 110 may be configured to slide onto the limb 202 and may be secured in place once the bladder 206 is inflated. In other embodiments, the cuff 110 may be substantially rectangular and may be configured to be wrapped around the limb 202. The cuff 110 may be secured in place on the limb 202 by connecting a first end of the cuff 110 to a second end of the cuff 110 with any type of fastener that enables the cuff 110 to be secured around the limb 202. For example, a hook and latch system can be used in some embodiments.

In order to achieve the desired blood flow restriction, the cuff 110 may be about 15 cm wide. In other embodiments, the cuff 110 may be less than 15 cm wide or greater than 15 cm wide depending on the desired application. The wider the cuff 110, the more distributed the pressure may be across the limb 202 which may lead to a higher pressure being delivered to the limb 302 to achieve the desired blood flow restriction. The width of the cuff 110 may also be varied to increase patient comfort while completing the blood flow restrictive therapy. Different embodiments of the cuff 110 may be different lengths depending on the desired application. For example, a cuff designed to be worn on a leg may be longer than a cuff designed to be worn on an arm.

The cuff 110 may include the bladder 206. In some embodiments, the bladder 206 may be formed integrally with the cuff 110 such that it is located between the exterior surface 302 and the interior surface 304 of the cuff 110. In other embodiments, the bladder 206 may be a separate component that may be inserted into the cuff 110 between the exterior surface 302 and the interior surface 304. The bladder 206 may be configured to be fluidly coupled to the source of positive pressure 105. When actuated, the source of positive pressure 105 may deliver fluid through the conduit 204 into the bladder 206. When fluid flows into the bladder 206, the bladder 206 may swell or inflate.

Continuing with FIG. 3, the blood flow sensor 130 may be coupled to the interior surface 304 of the cuff 110 and may be disposed between the interior surface 304 and the limb 202. The blood flow sensor 130 may be coupled to the interior surface 304 of the cuff 110 by an adhesive, by a weld, or by another method of coupling that will ensure the blood flow sensor 130 remains in place on the interior surface 304 of the cuff 110. In some embodiments, the cuff 110 may further include a power source that may provide power to the blood flow sensor 130. The power source may be a rechargeable battery such as a lithium ion battery. In other embodiments, the power source may be another source that may provide power to the blood flow sensor 130. The blood flow sensor 130 may be positioned to sense blood flow through the veins and the arteries of the limb 202. In some embodiments, the blood flow sensor 130 may include a transmitter 306 and a receiver 308. The transmitter 306 can be an LED transmitter in some embodiments. In other embodiments, the transmitter 306 may be a laser transmitter or another transmitter that is adapted to generate a signal (S1) propagating towards the receiver 308 as indicated by dashed arrows 310 that impinge on the receiver 308 to determine the blood flow through the limb 202. In some embodiments, the receiver 308 may be a photodiode that can be configured to receive a light signal from the transmitter 306. In other embodiments, the receiver 308 may be another type of receiver that is configured to receive the signal (S1) from the transmitter 306. The blood flow sensor 130 is shown in FIG. 3 with the transmitter 306 and the receiver 308 juxtaposed on opposite sides of the interior surface 304 of the cuff 110. The signal (S1) may be emitted from the transmitter 306, may travel through the limb 202, and may be received by the receiver 308. In other embodiments, the transmitter 306 and the receiver 308 may be located at different locations on the interior surface 304 of the cuff 110.

In some embodiments, the blood flow sensor 130 may be a photoplethysmography (PPG) sensor and may include the transmitter 306 and the receiver 308. In other embodiments, the blood flow sensor 130 may be an optical sensor, such as an Optical Blood-Flow Sensor available from Kyocera, and may include the transmitter 306 and the receiver 308. In still other embodiments, the blood flow sensor 130 may be an ultrasonic flow sensor, a Doppler sensor, a transit time sensor, or another non-contact blood flow sensor that can measure the blood flow through the limb 202.

In some embodiments, the blood flow sensor 130 may further be configured to measure oxygen levels of blood flow through the arteries of the limb 202. For example, if the blood flow sensor 130 is a PPG sensor, it may be configured to determine the oxygen level of the blood flow through the arteries of the limb 202 as well as being configured to measure the blood flow through the veins and the arteries of the limb 202. In some embodiments, the oxygen level of the blood flow through the arteries of the limb 202 may be communicated to the user or a health care provider for monitoring. For example, the oxygen level of the blood flow through the arteries of the limb 202 may be monitored to ensure that the muscles in the limb 202 enter a hypoxia state to facilitate the release of growth hormones which may lead to muscle growth. In other embodiments, the oxygen level of the blood flow through the arteries of the limb 202 may be used to modify a target arterial blood flow or a target amount of pressure delivered from the source of positive pressure 105 to the bladder 206. For example, if the oxygen level of the blood flow through the arteries of the limb 202 is below a predetermined value, the pressure delivered by the cuff 110 to the limb 202 may need to be reduced to increase the blood flow through the arteries of the limb 202.

The cuff system 100 may further include the source of positive pressure 105. In some embodiments, the source of positive pressure 105 may be housed within the therapy unit 135. In other embodiments, the source of positive pressure 105 may be separate from the therapy unit 135 but may be communicatively coupled to the controller 115 of the therapy unit 135. The source of positive pressure 105 may be configured to deliver fluid through the conduit 204 to the bladder 206 of the cuff. In some embodiments, the source of positive pressure 105 may also be configured to remove fluid from the bladder 206 of the cuff 110. In some embodiments, the source of positive pressure 105 may be able to remove fluid from the bladder 206 automatically. In other embodiments, there may be a vent valve that may be configured to expose the bladder 206 to ambient environment surrounding the cuff system 100. The vent valve may allow fluid to be removed from the bladder 206, thereby reducing the pressure delivered by the bladder 206 to the limb 202.

In some embodiments, the source of positive pressure 105 may be an electric pump that is configured to deliver positive pressure to the bladder 206. In other embodiments, the source of positive pressure may be a manual pump that may be operated by a user or a health care provider to deliver positive pressure to the bladder 206 of the cuff 110.

In some embodiments, the therapy unit 135 may further include a user interface 315 communicatively coupled to the controller 115 for providing information about the cuff system 100. For example, the user interface 315 may be configured to output the pressure being delivered to the bladder 206 of the cuff 110 from the source of positive pressure 105. The user interface 315 may further be configured to output the blood flow levels through the veins and arteries of the limb 202 as sensed by the blood flow sensor 130.

Continuing with FIG. 3, the cuff system 100 is shown with the bladder 206 of the cuff 110 in a resting or a deflated state. The cuff 110 may be in the deflated state before it is secured around the limb 202. When deflated and secured around the limb 202, the blood flow sensor 130 may detect a baseline blood flow through the limb 202. The baseline blood flow may be the blood flow detected through the veins and the arteries of the limb 202 when there is no pressure applied to the limb 202 from the cuff 110.

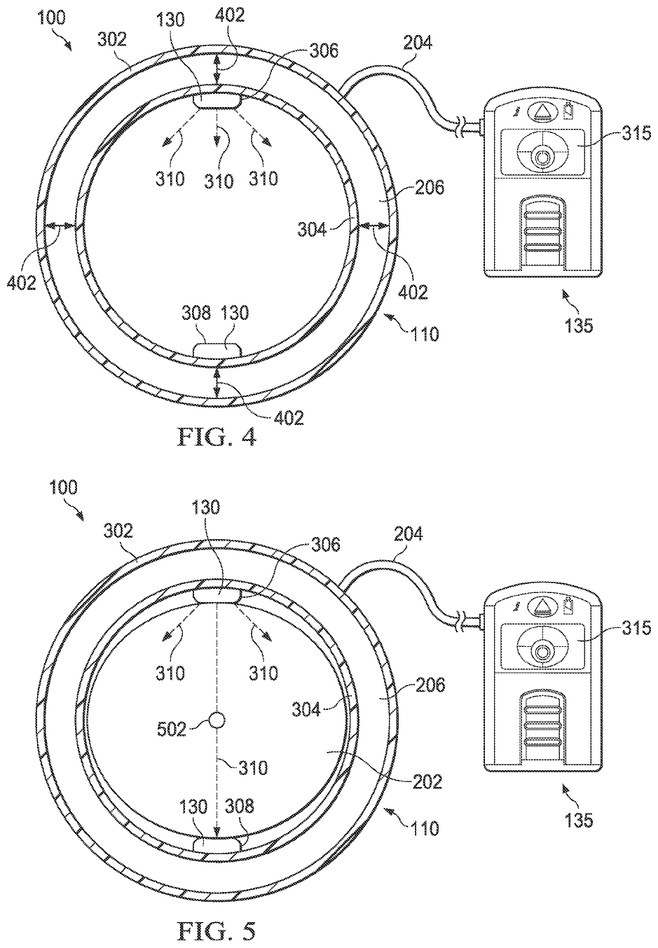

Referring to FIG. 4, the cuff system 100 of FIGS. 3 is shown with the bladder 206 of the cuff 110 in an inflated state. The bladder 206 may be fluidly connected to the source of positive pressure 105 through the conduit 204. The source of positive pressure 105 may deliver fluid through the conduit 204 to the bladder 206. When fluid enters the bladder 206, the bladder 206 may begin to inflate as indicated by bidirectional arrows 402. While the bladder 206 is being inflated, the volume of the bladder 206 may increase and the distance between the exterior surface 302 and the interior surface 304 of the cuff 110 may increase.

When the cuff 110 is applied around the limb 202 of a user, the cuff 110 may apply pressure to the limb 202 when the bladder 206 is inflated. The blood flow sensor 130 may be configured to continuously monitor blood flow through the veins and the arteries of the limb 202 as the bladder 206 is inflated. The blood flow sensor 130 may be communicatively coupled to the controller 115 and the controller 115 may be configured to receive signals from the blood flow sensor 130. The controller 115 may be communicatively coupled to the user interface 315 of the therapy unit 135 and the user interface 315 may be configured to output information about the blood flow, e.g., flow rate and oxygen level, through the veins and arteries of the limb 202. A user or health care provider may use the therapy unit 135 and the user interface 315 to monitor the cuff system 100 during blood flow restrictive therapy.

In operation, the blood flow sensor 130 may measure a baseline blood flow through the veins and the arteries of the limb 202 before the bladder 206 is inflated. As the bladder 206 begins to inflate, the cuff 110 will begin to exert a force or a pressure on the limb 202. The cuff 110 may first begin to restrict blood flow through veins of the limb 202. As the bladder 206 is further inflated, the force or pressure exerted on the limb 202 by the cuff 110 may be increased. As the pressure exerted on the limb 202 is increased, blood flow through the veins of the limb 202 may be completely occluded and blood flow through the arteries of the limb 202 may begin to be restricted. The blood flow sensor 130 may continue to monitor the blood flow through the arteries of the limb 202 as the bladder 206 is inflated. The bladder 206 may continue to be inflated until the blood flow sensor 130 senses that the blood flow through the arteries of the limb 202 is completely occluded. When the blood flow sensor 130 senses that the blood flow through the arteries of the limb 202 has been completely occluded, the cuff 110 is exerting a limb occlusion pressure (LOP) on the limb 202. The blood flow sensor 130 may send a signal to the controller 115 to indicate that the blood flow through the arteries of the limb 202 has reached the LOP which may be displayed on the user interface 315.

In some embodiments, the controller 115 may be communicatively coupled to the source of positive pressure 105 and the blood flow sensor 130 such that a feedback loop is formed between the controller 115, the source of positive pressure 105, and the blood flow sensor 130. The feedback loop may be further informed by inputs from a user, a health care provider, or a trainer. For example, a user, a health care provider, or a trainer may input therapy input values to the therapy unit 135. In some embodiments, the therapy input values may include a duration of positive pressure application and a desired amount of blood flow restriction. In other embodiments, the therapy input values may vary based on the patient using the cuff 110 and the specific physical therapy being completed. In some embodiments, the therapy input values may include frequency, load, restriction time, type, sets, cuff, repetitions pressure, rest between sets, restriction form, execution speed, and execution. The therapy input values may be input into the therapy unit 135 before the source of positive pressure 105 begins to deliver positive pressure to the bladder 206 of the cuff 110. For example, a user, a health care provider, or a trainer may input 15 minutes for the duration of positive pressure application or the restriction time and may input 50% for the desired amount of blood flow restriction which may allow the controller 115 to calculate the target arterial blood flow. With these inputs, the controller 115 may operate the cuff system 100 to reduce the arterial blood flow by 50% and may maintain the positive pressure in the bladder 206 of the cuff 110 for 15 minutes after the target arterial blood flow is reached. In some embodiments, the cuff system 100 may further have built in over-pressure and duration protection. For example, the controller 115 may be configured to deflate the cuff 110 if the pressure delivered to the bladder 206 of the cuff 110 is too high. For example, if the pressure being exerted on the limb 202 is higher than a predetermined threshold pressure, the controller 115 may be configured to deflate the bladder 206 of the cuff 110. The controller 115 may further be configured to deflate the cuff 110 if the bladder 206 of the cuff 110 has been inflated for too long. For example, if the bladder 206 of the cuff 110 has been inflated for longer than a predetermined threshold time the controller 115 may be configured to deflate the bladder 206 of the cuff 110.

Once the controller 115 receives a signal from the blood flow sensor 130 indicating that blood flow through the arteries of the limb 202 has reached the LOP, the controller 115 may stop the source of positive pressure 105 to stop fluid flow to the bladder 206. The controller 115 may then reverse the source of positive pressure 105 so that fluid is removed from the bladder 206. In other embodiments, the controller 115 may open the vent valve or may release fluid from the bladder 206 by another method to relieve the pressure being delivered to the limb 202. The controller 115 may release fluid from the bladder 206 until a target arterial blood flow is sensed by the blood flow sensor 130. The target arterial blood flow may be determined as a percentage between the baseline arterial blood flow and the LOP. In other embodiments, the controller 115 may release fluid from the bladder 206 until a predetermined pressure is being delivered from the source of positive pressure 105 to the bladder 206. For example, a first pressure may occur when there is no pressure being delivered to the bladder 206. A second pressure may be the LOP which may be the pressure being delivered to the bladder 206 of the cuff 110 when the LOP has been reached. The predetermined pressure may be a percent reduction from the second pressure so that blood flow resumes through the arteries of the limb 202 but is still restricted from the baseline arterial blood flow by the bladder 206 of the cuff 110 compared to the baseline arterial blood flow through the limb 202.

In other embodiments, the controller 115 may be communicatively coupled to the blood flow sensor 130 but may not be communicatively coupled to the source of positive pressure 105. The user interface 315 may be configured to output data about the blood flow through the veins and the arteries of the limb 202. A user or a health care provider may manually adjust the source of positive pressure 105 in response to the data that is output on the user interface 315 about the blood flow through the veins and arteries of the limb 202. The user or health care provider may monitor the cuff system 100 as the user interface 315 outputs that blood flow through the veins of the limb has stopped. In some embodiments, the cuff system 100 may also have built in over-pressure and duration protection to ensure that the cuff 110 is not applying more than a threshold amount of pressure to the limb 202 and that the cuff 110 is not exerting pressure on the limb 202 for longer than a threshold time. When the user interface 315 outputs a value or signal indicating that the LOP has been reached, the user or the health care provider may manually stop the source of positive pressure 105 from delivering positive pressure to the bladder 206 of the cuff 110. The user or health care provider may then initiate the release fluid from the bladder 206 of the cuff 110. For example, in some embodiments, the user or the health care provider may switch the source of positive pressure 105 to a vacuum mode to remove fluid from the bladder 206. In other embodiments, the user or the health care provider may open the vent valve to release fluid from the bladder 206 of the cuff 110. The user or the health care provider may stop the release of fluid from the bladder 206 when the target arterial blood flow is reached. The user or the health care provider become aware that the target arterial blood flow has been reached by an indication from the user interface 315 of the therapy unit 135. For example, the user interface 315 can output a value of the arterial blood flow through the limb 202. In other embodiments, the user interface 315 may output a sound, may vibrate, or may make another indication that the target arterial blood flow has been reached.

In any of the above embodiments, once the target arterial blood flow has been reached, a blood flow restrictive therapy may be started. The cuff 110 may deliver a constant pressure on the limb 202 such that the target arterial blood flow can be maintained for the duration of the blood flow restrictive therapy. After the blood flow restrictive therapy has been completed, the remaining fluid may be released from the bladder 206 of the cuff 110 in order to enable the cuff 110 to be removed from the limb 202.

Referring to FIG. 5, the cuff system 100 is shown in an operational state on the limb 202. The interior surface 304 of the cuff 110 is coupled to the limb 202 such that the blood flow sensor 130 is disposed between the interior surface 304 of the cuff 110 and the limb 202.The blood flow sensor 130 may be a PPG sensor and may include the transmitter 306 and the receiver 308. The blood flow sensor 130 may be in an absorptive mode with the receiver 308 disposed on an opposite side of the interior surface 304 as the transmitter 306. Thus, the signal (S1) from the transmitter 306 must travel through the limb 202 to reach the receiver 308.

A blood vessel 502 may be representative of the blood flow through the arteries of the limb 202. The signal (S1) generated by the transmitter 306 may propagate through the limb 202 and the blood vessel 502 to impinge on the receiver 308. The signal (S1) may be at least partially absorbed by the blood vessel 502 and may continue through the tissue of the limb 202 to impinge on the receiver 308. The signal (S1) is modified by both the tissue of the limb 202 and the blood vessel 502 so that the receiver 308 senses a receiver signal (S2) indicative of the different characteristics of both the tissue of the limb 202 and the blood vessel 502. For example, if the signal (S1) from the transmitter 306 is light, the light may be absorbed by the hemoglobin in the blood vessel 502. The wavelength of the light received by the receiver 308 may be modified so that the receiver signal (S2) indicates the amount of absorption that occurred at the blood vessel 502. The receiver signal (S2) may be interpreted to determine the arterial blood flow through the limb 202.

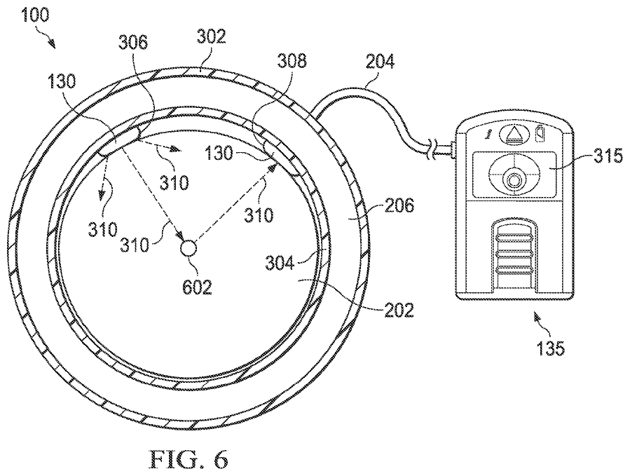

Referring to FIG. 6, the cuff system 100 is shown in an operational state on the limb 202. The interior surface 304 of the cuff 110 is coupled to the limb 202 such that the blood flow sensor 130 is disposed between the interior surface 304 and the limb 202. The blood flow sensor 130 may be an optical sensor and may include the transmitter 306 and the receiver 308. The blood flow sensor 130 may be in a reflectance mode with the receiver 308 disposed on the same side of the interior surface 304 as the transmitter 306. Thus, the signal from the transmitter 306 must travel partially though the limb 202 to contact the blood flow through the limb 202. Once the signal has reached the artery running through the limb 202, it may be reflected from the arterial blood flow and may be received by the receiver 308.

A blood vessel 602 may be representative of the blood flow through the arteries of the limb 202. The signal (S1) may be transmitted from the transmitter 306 and may flow through the limb 202 and the blood vessel 602. The signal (S1) may be reflected from the blood vessel 602 and may flow through the limb 202 to impinge on the receiver 308. Similar to that described above with reference to FIG. 5, the receiver signal (S2) received by the receiver 308 may have different characteristics that when the signal (S1) was emitted by the transmitter 306. For example, if the signal (S1) is light, the light may be absorbed by the hemoglobin in the blood vessel 602. The wavelength of the light received by the receiver 308 may indicate the amount of absorption that occurred at the blood vessel 602. The receiver signal (S2) received by the receiver 308 may be interpreted to determine the arterial blood flow through the limb 202.

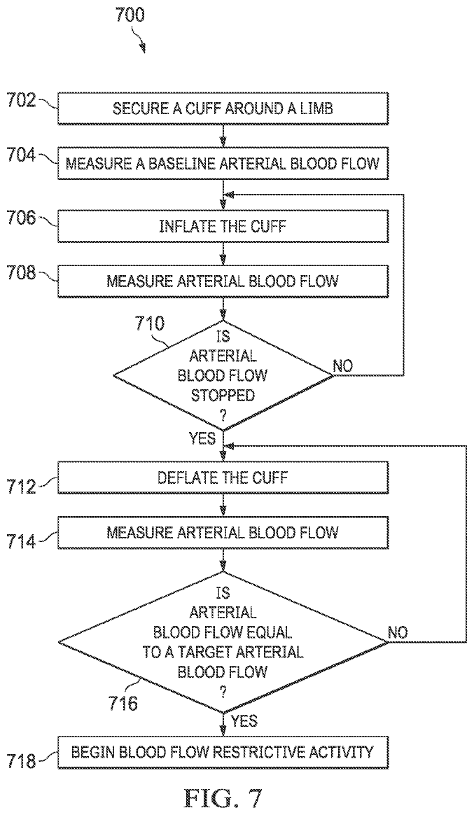

Referring primarily to FIG. 7 and to features previously described in FIGS. 1-5, a method 700 of restricting blood flow through arteries of the limb 202 is shown. In other embodiments, the method 700 may include additional, fewer, and/or different steps. Securing the cuff 110 around the limb 202 may be a first step such as step 702. The cuff 110 may include the bladder 206 and the blood flow sensor 130. The method 700 may continue with measuring a baseline arterial blood flow in step 704. The baseline arterial blood flow may be measured by the blood flow sensor 130 when the cuff 110 is in a resting state where the bladder 206 is not inflated. In step 706, the cuff 110 may be inflated. The cuff 110 may be inflated by inflating the bladder 206 with the source of positive pressure 105. In step 708, the arterial blood flow may be measured again. The arterial blood flow may be measured by the blood flow sensor 130 of the cuff 110. In some embodiments, the blood flow sensor 130 may be continuously monitoring the arterial blood flow through the limb 202 as the cuff 110 is inflated and in other steps it may only be monitored at the step 708.

Continuing with the method 700, conditional step 710 assesses whether arterial blood flow through the limb 202 has stopped. If the blood flow sensor 130 senses blood flow through the limb 202, then arterial blood flow through the limb 202 has not stopped. The method 700 will then return to step 706 and the cuff 110 will continue to be inflated. If the blood flow sensor 130 does not sense blood flow through the limb 202, then arterial blood flow has stopped and the method 700 will move on to step 712. In step 712 the cuff 110 is deflated. The cuff 110 is deflated to allow arterial blood flow to resume flowing through the limb 202. In step 714 the arterial blood flow is measured again. Similar to step 708, the blood flow sensor 130 of the cuff 110 may sense the arterial blood flow through the limb 202. The blood flow sensor 130 may continuously sense the arterial blood flow or may measure the arterial blood flow only at the step 706 and the step 708.

Continuing with the method 700, conditional step 716 assesses whether the arterial blood flow is equal to a target arterial blood flow. The target arterial blood flow may be an arterial blood flow level between the baseline arterial blood flow and when the LOP. The target arterial blood flow may be a predetermined blood flow level through the arteries of the limb 202 that may be determined by a health care professional. If the arterial blood flow measured in step 714 is not equal to the target arterial blood flow, the method 700 may return to step 712. The cuff 110 may continue to be deflated and the blood flow sensor 130 may monitor the blood flow through the limb 202 until the target arterial blood flow is reached. Once the target arterial blood flow is reached, the method 700 may continue to step 718. In step 718, a blood flow restrictive activity may be performed. The blood flow restrictive activity may not be performed until the target arterial blood flow is reached because it may not be desirable to perform the blood flow restrictive activity when there is too little blood flow or too much blood flow through the limb 202. The target arterial blood flow may be a predetermined blood flow that may be determined by a health care professional to be safe for a patient or a user to complete the blood flow restrictive activity.

Other methods for restricting blood flow through the arteries of the limb 202 are provided. In some examples, the method may include providing the cuff 110. The cuff 110 may include the interior surface 304, the bladder 206, and the blood flow sensor 130. The interior surface 304 of the cuff 110 may be secured around the limb 202. The bladder 206 may be configured to be fluidly coupled to the source of positive pressure 105 and to restrict blood flow through the arteries of the limb 202 when inflated. The blood flow sensor 130 may be coupled to the interior surface 304 and may be disposed between the interior surface 304 and the limb 202. The blood flow sensor 130 may be configured to measure blood flow through the arteries of the limb.

The method may further include securing the cuff 110 around the limb 202, sensing, with the blood flow sensor 130, a baseline arterial blood flow through the arteries of the limb 202, and coupling the bladder 206 to the source of positive pressure 105. The method may further include inflating the bladder 206 with the source of positive pressure 105 until the blood flow sensor 130 senses a restricted arterial blood flow. In some embodiments, the restricted arterial blood flow may indicate that the blood flow through the arteries of the limb is completely occluded. The method may further include adjusting the bladder 206 to reach a target arterial blood flow. The target arterial blood flow may be between the baseline arterial blood flow and the restricted arterial blood flow.

In some embodiments, adjusting the bladder 206 to reach a target arterial blood flow may include releasing fluid from the bladder 206 of the cuff 110 until the target arterial blood flow is reached. In other embodiments, adjusting the bladder 206 to reach a target arterial blood flow may include receiving, with the controller 115, a signal from the blood flow sensor 130 that the restricted arterial blood flow has been reached. Adjusting the bladder 206 to reach the target arterial blood flow can further include sending, with the controller 115, a signal to the source of positive pressure 105 to stop the source of positive pressure 105 from delivering positive pressure to the bladder 206 of the cuff 110. Adjusting the bladder 206 to reach the target arterial blood flow can further include releasing fluid from the bladder 206 of the cuff 110 until the target arterial blood flow is reached. In some embodiments, releasing fluid from the bladder 206 of the cuff 110 until the target arterial blood flow is reached can include sending, with the controller 115, a signal to the source of positive pressure 105 to switch the source of positive pressure to a vacuum mode and removing fluid from the bladder 206 of the cuff 110 with the source of positive pressure 105 until the target arterial blood flow is reached.

In some example embodiments, the method can further include sensing, with the blood flow sensor 130, an oxygen level of blood in the arteries of the limb 202. The method can further include modifying the target arterial blood flow to incorporate the oxygen level of blood in the arteries of the limb 202.

The systems, apparatuses, and methods described herein may provide significant advantages. For example, the cuff system 100 may continuously monitor the blood flow through the limb 202 while the cuff 110 is restricting the blood flow through the limb 202 and during a blood flow restrictive activity. Having continuous blood flow readings may allow health care providers to optimize the pressure delivered from the cuff 110 to the limb 202 which may optimize therapy for the end user. In some embodiments, the blood flow through the limb 202 may inform a feedback loop which may control the pressure delivered from the cuff 110 to the limb 202. The cuff 110 may be adjusted manually or automatically to achieve the optimal therapy pressure for the end user. In some embodiments, the cuff system 100 may also provide real time oxygen level readings which may allow health care providers to further optimize the pressure delivered from the cuff 110 to the limb 202.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may also be combined or eliminated in various configurations for purposes of sale, manufacture, assembly, or use. For example, in some configurations the controller 115 may be manufactured, configured, assembled, or sold independently of other components.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

1. A cuff for measuring blood flow through arteries of a limb, comprising:
an interior surface configured to be secured around the limb;
a bladder configured to be fluidly coupled to a source of positive pressure, and adapted to inflate in response to application of a positive pressure and restrict blood flow through the arteries of the limb when inflated; and
a blood flow sensor coupled to the interior surface and disposed between the interior surface and the limb, the blood flow sensor configured to measure blood flow through the arteries of the limb.

2. The cuff of claim 1, wherein the blood flow sensor is a PPG sensor having an LED and a photodiode.

3. The cuff of claim 2, wherein the PPG sensor is in an absorptive mode with the LED and the photodiode disposed on opposite sides of the interior surface of the cuff.

4. The cuff of claim 2, wherein the PPG sensor is in a reflectance mode with the LED and the photodiode aligned on one side of the interior surface of the cuff.

5. The cuff of claim 2, wherein the PPG sensor is configured to measure an oxygen level of blood in the limb.

6. The cuff of claim 1, wherein the blood flow sensor is an optical sensor having an LED and a photodiode.

7. The cuff of claim 6, wherein the optical sensor is in a reflectance mode with the LED and the photodiode aligned on one side of the interior surface of the cuff.

8. The cuff of claim 1, wherein the source of positive pressure is adjusted manually.

9. The cuff of claim 1, wherein the blood flow sensor is communicatively coupled to a controller, the controller being configured to receive signals from the blood flow sensor.

10. The cuff of claim 9, wherein the controller is further configured to be communicatively coupled to the source of positive pressure and to adjust the source of positive pressure in response to the signals received from the blood flow sensor.

11. A system for measuring blood flow through arteries of a limb, the system comprising:
a cuff comprising:
an interior surface configured to be secured around the limb;
a bladder adapted to inflate in response to application of a positive pressure and restrict blood flow through the arteries when inflated; and
a blood flow sensor coupled to the interior surface and disposed between the interior surface and the limb, the blood flow sensor configured to measure blood flow through the arteries of the limb; and
a source of positive pressure configured to be fluidly coupled to the bladder of the cuff and to inflate the bladder of the cuff when actuated.

12. The system of claim 11, further comprising a controller communicatively coupled to the blood flow sensor and the source of positive pressure, the controller configured to receive signals from the blood flow sensor and to actuate the source of positive pressure in response to the signals from the blood flow sensor.

13. The system of claim 12, wherein the controller is further configured to receive therapy input values and to actuate the source of positive pressure in response to the therapy input values.

14. The system of claim 11, further comprising a therapy unit configured to output a value of the blood flow through the arteries of the limb.

15. The system of claim 11, wherein the blood flow sensor is further configured to measure an oxygen level of blood in the arteries of the limb.

16. A method of restricting blood flow through arteries of a limb, the method comprising:
providing a cuff comprising:
an interior surface configured to be secured around the limb;
a bladder configured to be fluidly coupled to a source of positive pressure, and adapted to inflate in response to application of a positive pressure and restrict blood flow through the arteries of the limb when inflated; and
a blood flow sensor coupled to the interior surface and disposed between the interior surface and the limb, the blood flow sensor configured to measure blood flow through the arteries of the limb;
securing the cuff around the limb;
sensing, with the blood flow sensor, a baseline arterial blood flow through the arteries of the limb;
coupling the bladder to the source of positive pressure;
inflating the bladder with the source of positive pressure until the blood flow sensor senses a restricted arterial blood flow, the restricted arterial blood flow indicating that the blood flow through the arteries of the limb is completely occluded; and
adjusting the bladder to reach a target arterial blood flow, the target arterial blood flow being between the baseline arterial blood flow and the restricted arterial blood flow.

17. The method of claim 16, wherein adjusting the bladder to reach the target arterial blood flow comprises:
receiving, with a controller, a signal from the blood flow sensor that the restricted arterial blood flow has been reached;
sending, with the controller, a signal to the source of positive pressure to stop the source of positive pressure from delivering positive pressure to the bladder of the cuff; and
releasing fluid from the bladder of the cuff until the target arterial blood flow is reached comprising:
sending, with the controller, a signal to the source of positive pressure to switch the source of positive pressure to a vacuum mode; and
removing fluid from the bladder of the cuff with the source of positive pressure until the target arterial blood flow is reached.

18. The method of claim 16, wherein adjusting the bladder to reach the target arterial blood flow comprises releasing fluid from the bladder of the cuff until the target arterial blood flow is reached.

19. The method of claim 16, further comprising:
sensing, with the blood flow sensor, an oxygen level of blood in the arteries of the limb; and
modifying the target arterial blood flow to incorporate the oxygen level of blood in the arteries of the limb.

20. (canceled)
